(19) United States
(12) Patent Application Publication
(10) Pub. No.: US 2025/0248783 A1
(43) Pub. Date: Aug. 7, 2025

(54) THREE-AXIS ACTUATOR FOR A PORTABLE MICROSCOPE

(71) Applicants: Sam Powdrill, Wilmore, KY (US); Sam Pletcher, Indianapolis, IN (US)

(72) Inventors: Sam Powdrill, Wilmore, KY (US); Sam Pletcher, Indianapolis, IN (US)

(21) Appl. No.: 19/191,001

(22) Filed: Apr. 28, 2025

Related U.S. Application Data
(63) Continuation-in-part of application No. 17/567,849, filed on Jan. 3, 2022, now Pat. No. 12,313,832.

(51) Int. Cl. A61B 90/25; G02B 21/24

(52) U.S. Cl. CPC A61B 90/25; G02B 21/24

(57) ABSTRACT

A motion control system is disclosed for enabling three-axis positioning of an optical device, such as a portable microscope. The system includes an XY actuator module for lateral movement in orthogonal planar directions, a Z actuator module for vertical displacement to adjust focus, and a wireless control interface. The wireless control interface comprises a joystick and momentary switches, allowing independent, hands-free control of all three axes via radio frequency signals. The actuator modules are modular, lightweight, and powered by batteries or external power sources, making the system highly portable and suitable for field or surgical applications. The Z actuator adjusts only the objective lens, reducing weight and enabling micro-level focal changes without moving the entire microscope body. The system offers precision, stability, and ease of deployment, improving user ergonomics and operational efficiency in both clinical and mobile environments.

### CROSS-REFERENCE TO RELATED APPLICATIONS

The present application is a continuation-in-part of U.S. application Ser. No. 17/567,849, filed 3 Jan. 2022, which claims priority to and the benefit of U.S. Provisional Application No. 63/146,661, filed on Feb. 7, 2021, the contents of which are incorporated herein by reference in their entirety.

### FIELD OF THE INVENTION

The present invention disclosed herein generally relates to actuators. More particularly, the present invention discloses a three-axis actuator for a portable microscope. The disclosed actuator allows foot control of the three axes of movement of the portable microscope for use in various applications such as in surgery in a mobile setting.

### BACKGROUND

Robotic assemblies for surgery or microsurgery comprising microscope terminating with surgical instruments are known in the field. Solutions available in the state-of-the-art, require a motion strategy that simultaneously involves movements even for small motions of the microscopic instrument in the operating work-field, which results both in a difficult control of the kinematic accuracy and in a large encumbrance in the operating work-field, that in practice becomes inaccessible to the surgeon. As a matter of fact, the application field of the majority of robotic assemblies for microscopic surgery are dedicated to use in minimally invasive surgery (or MIS), such as laparoscopic or endoscopic surgery. In both such applications, the kinematics of the robotic assembly is aimed to optimize the access of the surgical instruments to the operating field through the surgical ports or orifices, a feat that requires the coordination of a plurality of degrees of freedom of movement. In contrast, surgical, and microsurgical, applications in open surgery require an accurate kinematic control of translational movements, over a workspace limited by the field of view of the operating microscope, without the limiting kinematic constraints represented by the surgical ports or natural orifices, and thus benefit hugely from the surgeon's ability to directly access the operating field. Thus, it becomes important to efficiently control the movement of the microscope within the field of view. Generally, actuators are used to control the movement of the microscope. Microscopes have had 3 axis actuators on them from many years. However, the primary issues with them for a portable microscope is that they are developed for very heavy microscopes. There is no prior art that explicitly discloses easily portable 3 axis actuators for the microscopes. Therefore, the purpose of the present invention is to provide a three-axis actuator for a portable microscope for facilitating an efficient and effective movement of the portable microscope.

### SUMMARY

It will be understood that this disclosure is not limited to the apparatus described herein, as there can be multiple possible embodiments of the present disclosure which are not expressly illustrated in the present disclosure. It is also to be understood that the terminology used in the description is for the purpose of describing the particular versions or embodiments only and is not intended to limit the scope of the present disclosure.

In one aspect, the present invention provides a three-axis actuator for a portable microscope, which may allow foot control of the three axes to provide efficient and effective movement of the portable microscope for use in various applications, such as surgery in a mobile setting. The three-axis actuator for a portable ophthalmic microscope, especially intended for cataract surgery, although other uses are possible, addresses the challenge of microscope portability. The actuator may be adapted for travel by plane to enable use in remote areas. It is lightweight, stable, and designed to fit within a protective case, such as a Pelican case. In one exemplary embodiment, the case may have internal dimensions of approximately 22 inches by 17 inches by 10 inches and external dimensions of approximately 24.6 inches by 19.7 inches by 11.7 inches. To meet airline baggage weight restrictions, the maximum gross weight may be 50 pounds, with the case itself weighing approximately 20 pounds, thereby necessitating a total assembly weight of 30 pounds or less.

Design considerations for the actuator include X-Y actuation for centering the microscope over a patient's pupil. Actuation may be achieved via a foot pedal or joystick and is located below the microscope to reduce the overall height of the assembly, thereby increasing rigidity and reducing weight. In contrast, prior art often suspends the microscope below an X-Y actuation table, which increases height and decreases stability. The X and Y axes currently utilize approximately 100 mm (about 2 inches) of travel, which has been found sufficient for this application. The system employs double rails with integrated actuator motors to achieve this movement.

Z-axis actuation is also provided for adjusting the microscope's focal plane, which becomes increasingly narrow at higher magnifications. Z-axis movement may be actuated via a foot pedal, featuring separate pedals for upward and downward motion. The Z-axis actuator allows for approximately 25 mm of vertical travel and is housed within the system's base for compactness and protection.

The foot pedal itself is wireless, communicating via discrete radio frequencies rather than variable signals. Up to six discrete channels may be employed to control movement along the +X, −X, +Y, −Y, +Z, and −Z directions, with X-axis commands transmitted at 433 MHz and Y-axis commands transmitted at 315 MHz. The foot pedal joystick may produce motion in eight directions (north, northeast, east, southeast, south, southwest, west, and northwest), providing intuitive control. Unlike prior art foot pedals, which are typically large and heavy (approximately 10 pounds), the present foot pedal is lightweight and low-profile, measuring approximately 6 inches by 7 inches and weighing only about 8 ounces. Rubber footplates enhance grip and stability. Furthermore, the pedal's simplified RF communication design eliminates the need for pairing procedures common in prior systems.

To stabilize the assembly during use, a water bladder may be employed as ballast, providing stability without significantly increasing transport weight. Power for the actuators is supplied by either a 12V or 9V lithium battery, and an optional 5V USB power output port may be provided on the assembly's side to power auxiliary devices, such as a GoPro camera, if desired.

In one aspect, the present invention provides a motion control system for positioning an optical device. The motion control system comprises an XY actuator module configured to displace the optical device in a first planar direction and a second planar direction orthogonal to the first planar direction. The motion control system further comprises Z actuator module configured to displace at least a portion of the optical device in a direction perpendicular to the first and second planar directions. The motion control system further comprises a wireless control interface configured to transmit control signals to the XY actuator module and the Z actuator module to independently actuate each module.

In an embodiment, the XY actuator module comprises a pair of linear actuators and a platform for supporting the optical device.

In an embodiment, the Z actuator module is configured to adjust the position of a lens or objective component of the optical device to perform a focal adjustment.

In an embodiment, the wireless control interface comprises a joystick configured to control displacement along the planar directions and one or more momentary switches configured to control displacement along the perpendicular direction.

In an embodiment, at least one of the XY actuator module or the Z actuator module comprises a micro linear actuator responsive to wireless signals.

In an embodiment, the XY actuator module and the Z actuator module are mechanically decoupled and operable independently.

In an embodiment, the XY actuator module and the Z actuator module are modular and removably attachable to the optical device or a support structure coupled to the optical device.

In an embodiment, the actuator modules are powered by a portable battery housed within the system or the wireless control interface.

In an embodiment, the system enables repositioning and focal adjustment of the optical device without physical contact between a user and the optical device.

In an embodiment, each of the XY actuator module and the Z actuator module comprises a structural housing, an actuator motor, and a mounting interface configured to couple with the optical device or its support structure.

The disclosed motion control system provides several advantages over conventional microscope positioning solutions, particularly in applications requiring hands-free, precise, and modular manipulation. By integrating an XY actuator module and a Z actuator module, the system enables independent and accurate displacement of the microscope in all three spatial dimensions, allowing users to reposition the field of view and adjust focus without manual interference. The use of a wireless control interface, including a joystick and momentary switches, allows seamless and intuitive control from a remote location, ideal for surgical, diagnostic, or field applications where sterility or accessibility is critical. The modularity of the actuator components allows for easy attachment, removal, or replacement, increasing adaptability across various microscope models and setups. Furthermore, the compact, lightweight construction and low-power operation, including battery compatibility, make the system highly portable and suitable for mobile deployments. The invention significantly enhances operational efficiency, ergonomic safety, and user precision in microscope-based workflows.

These and other features and advantages of the present invention will become apparent from the detailed description below, in light of the accompanying drawings.

### BRIEF DESCRIPTION OF ACCOMPANYING DRAWINGS

The foregoing summary, as well as the following detailed description of the innovation, is better understood when read in conjunction with the appended drawings. For the purpose of illustrating the innovation, exemplary constructions of the innovation are shown in the drawings. However, the innovation is not limited to the specific methods and structures disclosed herein. The description of a method step or a structure referenced by a numeral in a drawing is applicable to the description of that method step or structure shown by that same numeral in any subsequent drawing herein.

FIG. 1 is a diagram that illustrates a foot pedal in an explored version, according to an exemplary embodiment of the present invention.

FIG. 2 is a diagram that illustrates an X-Y actuator in an explored version, according to an exemplary embodiment of the present invention.

FIG. 3 is a diagram that illustrates a Z actuator in an explored version, according to an exemplary embodiment of the present invention.

FIG. 4 is a diagram that illustrates an assembled foot pedal, according to an exemplary embodiment of the present invention.

FIG. 5 is a diagram that illustrates a use case scenario of using the three-axis actuator, according to an exemplary embodiment of the present invention.

FIG. 6 is an exploded perspective diagram that illustrates the sub-assembly of the actuator base system, showing various mechanical, electronic, and fastener components, according to an exemplary embodiment of the present invention.

FIG. 7 illustrates an exploded assembly view of a partially constructed actuation and linkage subsystem, which forms part of a three-axis actuator system for a portable microscope, according to an exemplary embodiment of the present invention.

FIG. 8 provides an exploded perspective view of the mechanical and control elements of the actuator subassembly, showing how upper rail structures, linkages, and mounting hardware interface with the electronic control system and linear motion platform, according to an exemplary embodiment of the present invention.

FIG. 9 illustrates an exploded assembly view of a compact user-interface module integrated atop the actuator platform, emphasizing the enclosure, control knob interface, and multi-layer structural shielding, according to an exemplary embodiment of the present invention.

FIG. 10 illustrates fully assembled setup of a three-axis actuator system designed for portable microscope manipulation, according to an exemplary embodiment of the present invention.

FIG. 11 illustrates a close-up view of the assembled control housing of the three-axis actuator system, according to an exemplary embodiment of the present invention.

FIGS. 12 and 13 illustrate integration of the three-axis actuator system with an optical microscope, according to an exemplary embodiment of the present invention.

Further areas of applicability of the present invention will become apparent from the detailed description provided hereinafter. It should be further understood that the detailed description of exemplary embodiments is intended for illustration purposes only and is, therefore, not intended to necessarily limit the scope of the invention.

### DETAILED DESCRIPTION

As used in the specification and claims, the singular forms “a”, “an”, and “the” may also include plural references. For example, the term “an article” may include a plurality of articles. Those with ordinary skill in the art will appreciate that the elements in the figures are illustrated for simplicity and clarity and are not necessarily drawn to scale. For example, the dimensions of some of the elements in the figures may be exaggerated, relative to other elements, to improve the understanding of the present invention. There may be additional components described in the foregoing application that are not depicted on one of the described drawings. In the event such a component is described, but not depicted in a drawing, the absence of such a drawing should not be considered as an omission of such design from the specification.

Before describing the present invention in detail, it should be observed that the present invention utilizes a combination of components, which contribute to a three-axis actuator for a portable microscope. The disclosed apparatus allows foot control of three axis of movement of the portable microscope for use in a surgery in a mobile setting. Accordingly, the components have been represented, showing only specific details that are pertinent for an understanding of the present invention so as not to obscure the disclosure with details that will be readily apparent to those with ordinary skill in the art having the benefit of the description herein. As required, detailed embodiments of the present invention are disclosed herein; however, it is to be understood that the disclosed embodiments are merely exemplary of the present invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the present invention.

References to “one embodiment”, “an embodiment”, “another embodiment”, “yet another embodiment”, “one example”, “an example”, “another example”, “yet another example”, and so on, indicate that the embodiment(s) or example(s) so described may include a particular feature, structure, characteristic, property, element, or limitation, but that not every embodiment or example necessarily includes that particular feature, structure, characteristic, property, element, or limitation. Furthermore, repeated use of the phrase “in an embodiment” does not necessarily refer to the same embodiment.

The words “comprising”, “having”, “containing”, and “including”, and other forms thereof, are intended to be equivalent in meaning and be open ended in that an item or items following any one of these words is not meant to be an exhaustive listing of such item or items or meant to be limited to only the listed item or items.

Unless stated otherwise, terms such as “first” and “second” are used to arbitrarily distinguish between the elements or entities. Thus, these terms are not necessarily intended to indicate temporal or other prioritization of such elements or priorities. While various exemplary embodiments of the disclosed apparatuses have been described below, it should be understood that they have been presented for purposes of example only, and not limitations. It is not exhaustive and does not limit the present invention to the precise form disclosed. Modifications and variations are possible in light of the below teachings or may be acquired from practicing of the present invention, without departing from the breadth or scope.

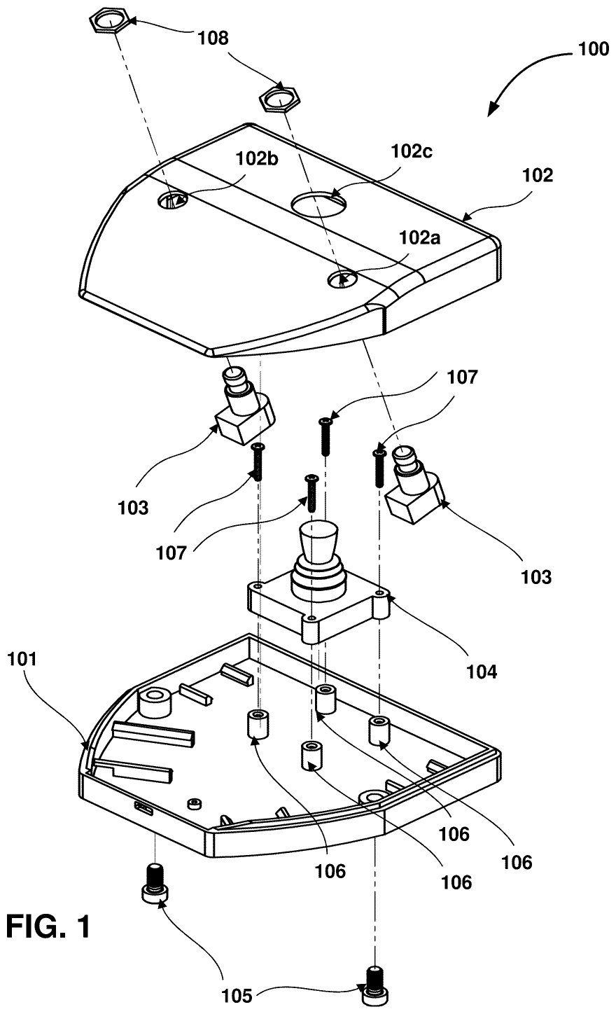

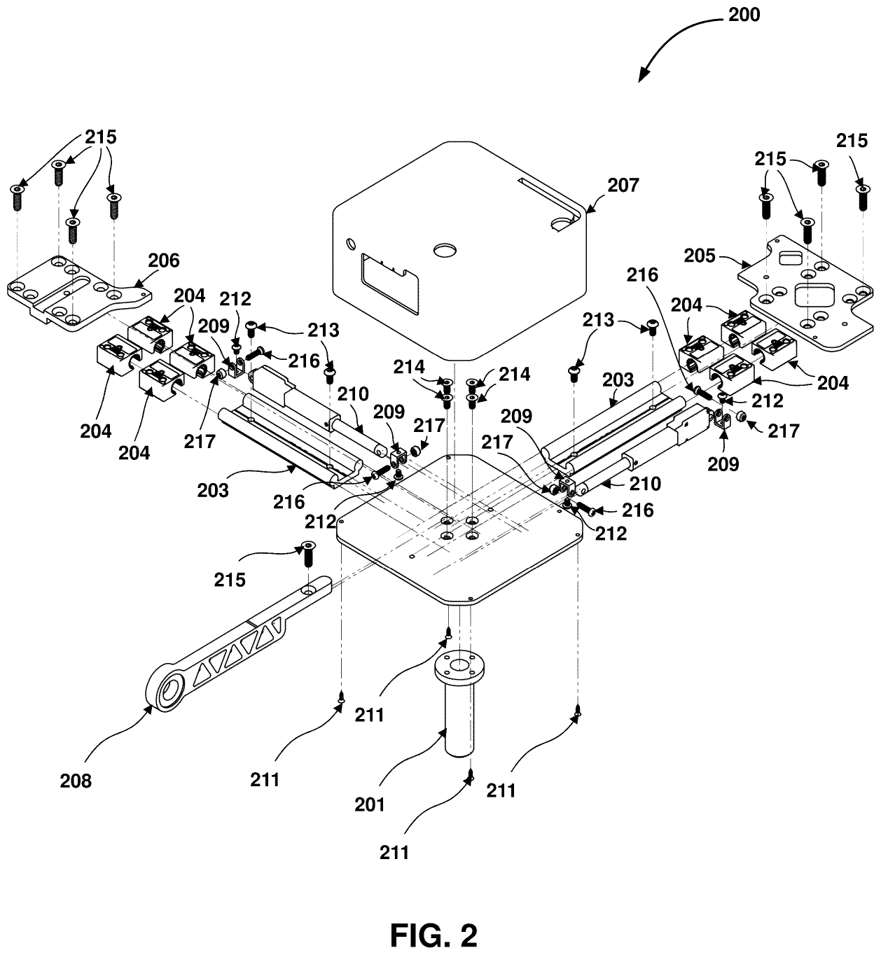

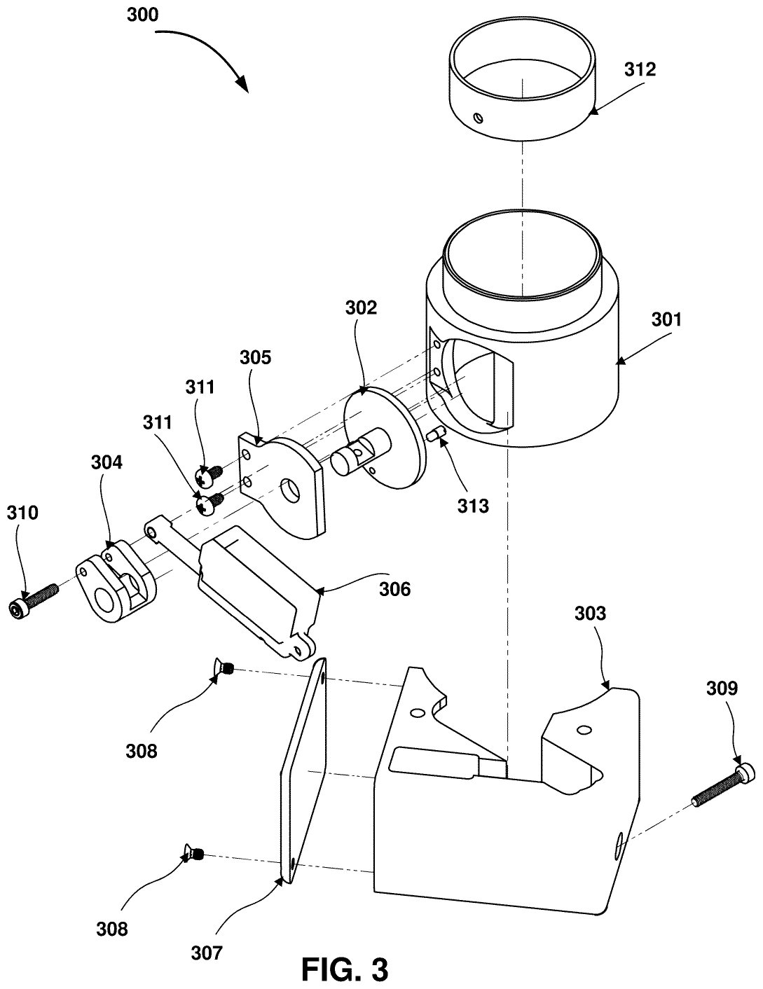

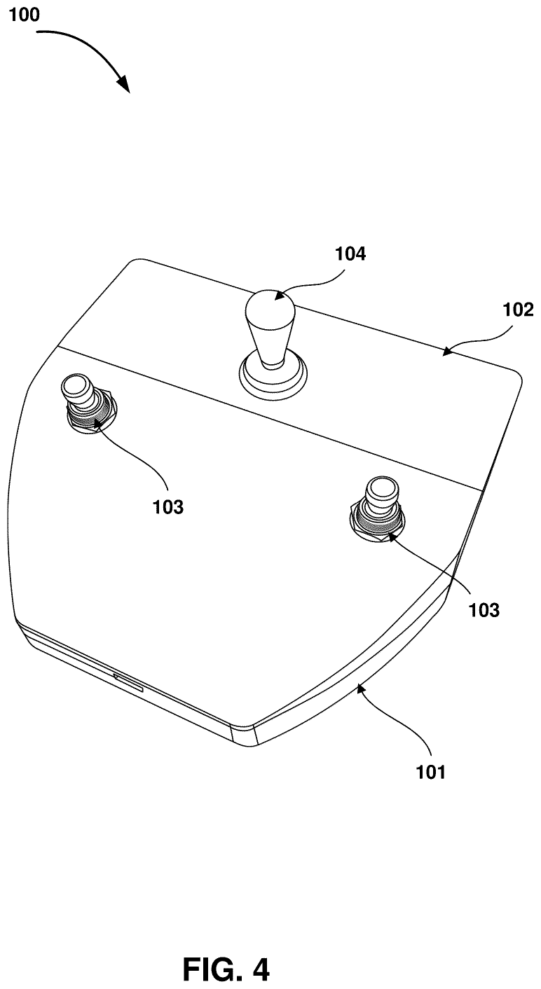

The three-axis actuator for a portable microscope of the present invention will now be described with reference to the accompanying drawings, which should be regarded as merely illustrative without restricting the scope and ambit of the present invention. Embodiments of the present invention will now be described with reference to FIGS. 1-5 to present a detailed overview of the three-axis actuator. An actuator is a device that produces a motion by converting energy and signals going into the system. The motion it produces can be either rotary or linear. The three-axis actuator produces motion along x-axis, y-axis, and z-axis. The three-axis actuator of the present invention primarily includes an XY actuator (shown in FIG. 2) and a Z actuator (shown in FIG. 3), which can be controlled using a foot pedal (shown in FIGS. 1 and 4). The present invention is meant to be modular in the sense that the XY actuator and the Z actuator may be used independently of each other. When the Z actuator is used by itself, a different simpler foot pedal with up and down control may be utilized. Generally, when the XY actuator is used, the Z actuator would be also (although not necessary) and in this case, the 3-axis foot pedal is used which uses a joystick for XY and 2 buttons to control the up and down of the Z actuator.

FIG. 1 is a diagram that illustrates a foot pedal 100 in an explored version, according to an exemplary embodiment of the present invention. The foot pedal 100 is configured to control one or more individual actuators using the radio frequency transmission. The foot pedal 100 includes a bottom portion 101 and a top portion 102 that act as enclosure of the foot pedal 100 when attached therebetween (as shown in FIG. 4). The top portion 102 includes three openings 102a, 102b, and 102c. The foot pedal 100 further includes two SPST (Single Pole Single Throw) momentary switches 103 that are configured to pass through two openings 102a and 102b in the top portion 102. The SPST momentary switch 103 is a push button that is used in applications which requires momentary ON or OFF switching action. Normally Open Push button switch are initially in OFF state as the contacts are not in contact with each, and when pushed down, the contacts gets closed and the path established between the two terminals of the push button. The foot pedal 100 further includes a joystick 104 that is configured to pass through the opening 102c in the top portion 102. The joystick 104 is a device consisting of a stick that pivots on a base and reports its angle or direction to the device it is controlling. The joystick 104 may be used to control the movement of one or more portable parts of the microscope. The foot pedal 100 further includes two M 6-14 mm socket heads 105. The socket head 105 is a screw that may be used to lock or attach the bottom portion 101 and the top portion 102 of the foot pedal 100 together. The foot pedal 100 further includes four rubber grips 106. The rubber grips 106 may provide gripping facilities to a respective component such as the joystick 104 when it is installed into the bottom portion 101. The foot pedal 100 further includes four M 2.5-20 mm Philips heads 107 and two SPST switch nuts 108.

In an embodiment, each SPST momentary switch 103 may be removably attached or connected to the top portion 102 by means of the SPST switch nut 108. Further, the joystick 104 may be removably attached or connected to the rubber grip 106 on the bottom portion 101 by means of the M 2.5-20 mm heads 107. The top portion 102 includes a circular hole (i.e., the opening 102c) from which the stick of the joystick 104 may come out and thus is easy operate. Further, the socket head 105 may be used to lock the bottom portion 101 and the top portion 102 of the foot pedal 100.

In an embodiment, for the foot pedal 100, the joystick 104 attaches to 4 radiofrequency transmitters to send signals to the XY actuator 200. The two momentary SPST buttons 103 attaches to the 2 radiofrequency transmitters to send signals to the Z actuator 300. Further, a rechargeable battery may be used to power the transmitters. In an embodiment, the foot pedal 100 may produce motion along 8 directions including North, North-East, East, South-East, South, South-West, West, and North-West.

FIG. 2 is a diagram that illustrates an X-Y actuator 200 in an explored version, according to an exemplary embodiment of the present invention. In an embodiment, the XY actuator 200 attaches to the microscope and stands and moves the microscope in the X and Y planes. The X-Y actuator 200 includes various components such as a top pin mount 201, a bottom plate 202, two linear rails 203, eight pillow blocks 204, a Y-axis plate 205, a top arm mount 206, a cover 207, and an arm 208. The X-Y actuator 200 further includes four actuator brackets 209, two linear actuators 210, four ⅜-inch flat head screws 211, four M 4×6 mm button heads 212, four M 5×10 mm button heads 213, four M 5×10 mm flat heads 214, nine M 6×20 mm flat heads 215, four M 4×16 mm Philips heads 216, and M 4 Nylock nut 217. In an embodiment, the top pin mount 201 is removably attached or connected to the bottom plate 202 from its bottom and is tighten by means of the four heads 214. Further, the cover 207 is used to cover the bottom plate 202 by means of the four head screws 211. Further, each end of the linear actuator 210 is inserted into the respective actuator bracket 209 and is locked by means of the head 216 and the nut 217. Another button head 212 may be inserted through the actuator bracket 209 to tighten the grip. Further, the pillow blocks 204 may be rolled over the linear rail 203. Further, the arm 208 may inserted into the linear rail 203 and is then tighten by the head 215. Another button heads 213 may be used to tighten the liner rail 203.

In an embodiment, the two linear rails 210 with the four pillow block couplers per rail have been provided to handle the torque placed by the microscope and to allow for 2 axis movement. Two linear actuators 210 are provided for 50 mm movement. An arm attached to one level (to arm mount) attaches to the microscope where two directions of movement is translated to the microscope. The base plate (bottom plate 202) and the attached top mount pin 201 attaches to the microscope stand. The individual linear rails 203 are attached to the bottom plate or Y axis plate 202 to allow movement in both directions. Two separate 2 relay receivers receive the signals from the foot pedal to move both linear actuators 210.

FIG. 3 is a diagram that illustrates a Z actuator 300 in an exploded version, according to an exemplary embodiment of the present invention. The Z actuator 300 moves the microscope objective lens in the Z plane for focusing the microscope. The Z actuator 300 may include various components such as a case 301, a pin 302, a main box 303, a camshaft 304, a pin bracket 305, a linear actuator 306, and a side cover 307. The Z actuator 300 may further include two M 3-5 mm flat heads 308, a M 3-20 mm socket head 309, a M 3-14 mm socket head 310, three M 3-6 mm machine screws 311, an inner lens 312, and a lens screw 313. In an embodiment, the pin 302 is inserted into the pin bracket 305 through an opening, and then, the arrangement is locked to an outer opening in the case by means of the screws 311. Further, the camshaft 304 and the linear actuator 306 are removably connected to each other by means of the head 310. Further, the inner lens 312 is placed inside the case and is tighten by means of the lens screw 313.

In an embodiment, for the Z actuator 300, the lens case holds an objective lens below the microscope. The metal pin which rotates with a screw for the objective lens may translate linear motion in one direction into a rotation which moves the objective lens up and down. The micro linear actuator attaches to the pin using a cam shaft to translate the linear to rotary motion. The main box is a housing unit which has cut-outs to house and seat the linear actuator and camshaft system. A radio frequency receiver receives a signal from the foot pedal and operates the linear actuator. The materials have been chosen primarily to reduce and optimize weight of the entire system. We have chosen aluminium pillow blocks over less expensive zinc pillow blocks to save weight within the XY actuator linear rails. The invention will not work if we utilize traditional ball bearing slides as the torque of the microscope will bind up the XY movement. In an embodiment, the actuators are powered by a 12-volt or 9-volt lithium battery. The actuators are lightweight and stable and are designed to fit in a case such as Pelican case. Internal dimensions of the case include 22 inches by 17 inches by 10 inches, and external dimensions of the case include 24.6 inches by 19.7 inches by 11.7 inches.

FIG. 4 is a diagram that illustrates an assembled foot pedal 100, according to an exemplary embodiment of the present invention. The foot pedal 100 includes the bottom portion 101 and the top portion 102. The foot pedal 100 further includes two SPST momentary switches 103. The SPST switch 103 is a switch that only has a single input and can connect only to one output. The SPST momentary switch 103 is a push button that is used in applications which requires momentary ON or OFF switching action. Normally Open Push button switch are initially in OFF state as the contacts are not in contact with each and when pushed down the contacts gets closed and the path established between the two terminals of the push button. The foot pedal 100 further includes a joystick 104. The joystick 104 is a device consisting of a stick that pivots on a base and reports its angle or direction to the device it is controlling. The joystick 104 may be used to control the movement of one or more portable parts of the microscope. In an embodiment, for the foot pedal, the joystick 104 attaches to 4 radiofrequency transmitters to send signals to the XY actuator 200. The two momentary SPST buttons 103 attaches to the 2 radiofrequency transmitters to send signals to the Z actuator 300. Further, a rechargeable battery may be used to power the transmitters.

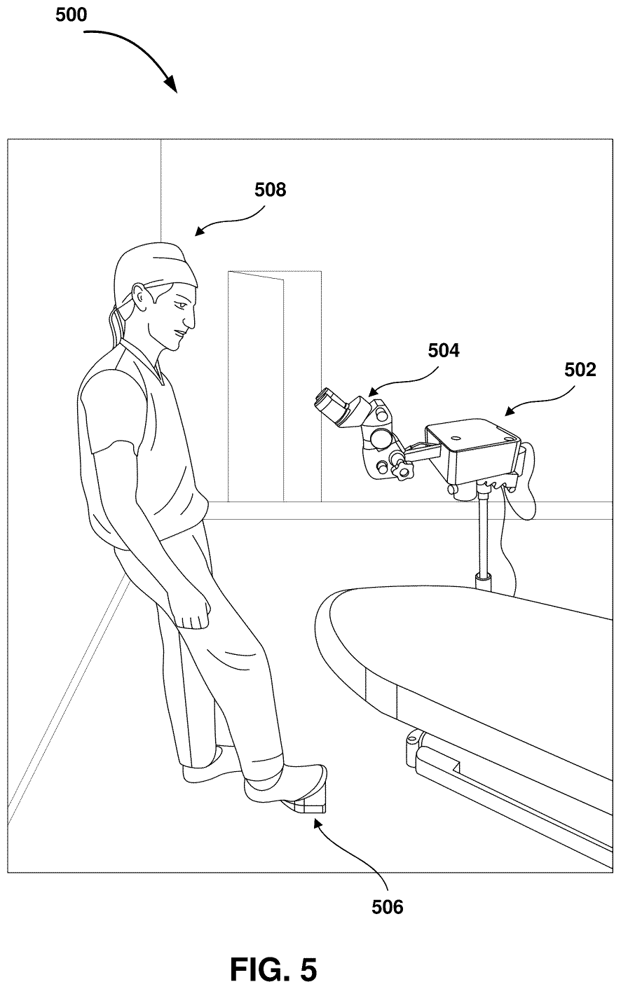

FIG. 5 is a diagram 500 that illustrates a use case scenario of using the three-axis actuator, according to an exemplary embodiment of the present invention. Here, as shown, a microscope 504 has been attached to the three-axis actuator 502. The three-axis actuator 502 may be configured to produce motion along x-axis, y-axis, and z-axis. For example, the XY actuator 200 (as shown in FIG. 2) may be configured to produce motion along x-axis and y-axis. The Z actuator 300 (as shown in FIG. 3) may be configured to produce motion along z-axis. These motion may be controlled by the foot pedal 506 (also shown by 100 in FIGS. 1 and 4). For example, an individual 508 (such as a doctor) may operate the foot pedal 506 to adjust and control the motion of the three-axis actuator 502 which in turn adjusts and control the motion of the microscope 502 along the three-axis. In an embodiment, the XY actuator attaches to the microscope and stand and moves the microscope in the X and Y planes. The Z actuator moves the microscope objective lens in the Z plane for focusing the microscope. The foot pedal controls the individual actuators using radio frequency transmission.

The disclosed 3-axis actuator differs in at least 3 distinct ways. Firstly, for the Z focus actuator, we have eliminated most of the weight of more traditional systems which raise and lower the microscope head by moving only the objective lens. This allows our invention to use a micro-linear actuator which is extremely light weight. Secondly, for the XY actuator, we believe we have a distinct design since all other XY actuators use a “dropdown” design whereas the microscope is directly underneath the XY actuator. The invention has a distinct design in that the XY actuator is located on the same level as the microscope and the design of our XY actuator had to accommodate sideways torque of the weight of the microscope. Thirdly, we believe we have a distinct difference in how the 3-axis actuator is powered in that we are running the entire 3 axis actuator simply with 9V of power and 1.4 mA of current allowing for our system to be run off of a battery and not need to be plugged into 110V.

The materials have been chosen primarily to reduce and optimize weight of the entire system. We have chosen aluminium pillow blocks over less expensive zinc pillow blocks to save weight within the XY actuator linear rails. Our invention will not work if we utilize traditional ball bearing slides as the torque of the microscope will bind up the XY movement.

Techniques consistent with the disclosure provide, among other features, the microscope actuators including one or more of the foot pedal 100, the XY actuator 200, and the Z actuator 300. While various exemplary embodiments of the disclosed unit have been described above, it should be understood that they have been presented for purposes of example only, and not limitations. It is not exhaustive and does not limit the disclosure to the precise form disclosed. Modifications and variations are possible in light of the above teachings or may be acquired from practicing of the disclosure, without departing from the breadth or scope. The present invention is meant to be modular in the sense that the XY actuator 200 and the Z actuator 300 may be used independently of each other. When the Z actuator 300 is used by itself, a different simple foot pedal with up and down control may be utilized. Generally, when the XY actuator 200 is used, the Z actuator 300 would also be used (although not necessary) and in this case, the 3-axis foot pedal is used which uses a joystick for XY actuator 200 and 2 buttons to control the up and down of the Z actuator 300.

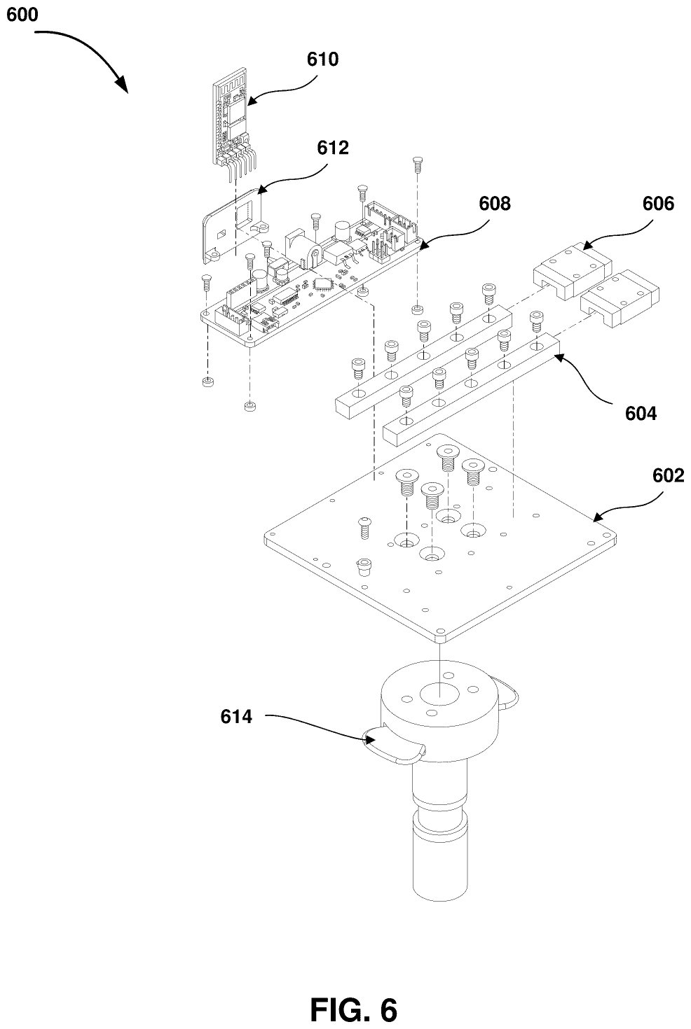

FIG. 6 illustrates an exploded perspective view 600 of an actuator subassembly configured for integration within a three-axis motion control system for a portable microscope. The subassembly includes a base plate 602, which serves as the primary structural platform upon which all mechanical and electronic components are mounted. Positioned above the base plate 602 are aluminum unthreaded spacers and spring mount spacers, which collectively function to elevate and isolate the upper structures from the base, thereby reducing vibrational coupling and mechanical strain. A pair of 9 mm linear rails 604 are mounted onto the upper face of the base plate 602, and are configured to support corresponding 9 mm carriages 606, enabling smooth linear translation along a defined axis for positioning applications. The electronics subsystem is represented by a printed circuit board (PCB) assembly 608, which is mounted on the elevated spacers and secured with various fasteners including M 3-6 mm machine screws and passivated flat head screws. This PCB interfaces with a Bluetooth module 610, such as an HC05 unit, to facilitate wireless communication between the actuator system and an external foot pedal controller. A PCB port cover 612 is also shown, which encloses and protects exposed circuitry and connectors during operation. A nylon plastic washer provides insulation and mechanical separation where needed between fastened surfaces.

Additionally, the subassembly includes a hangdown pin ratchet mechanism 614, which is mounted beneath the base plate 602 and serves as a mechanical linkage for vertical adjustment, rotation, or locking of the microscope mount or objective lens. The arrangement of these components demonstrates a modular and lightweight construction, with the use of precision fasteners and engineered spacers to ensure structural integrity, ease of assembly, and efficient serviceability. The configuration also reflects an emphasis on portability and field-readiness, aligning with the overall goal of providing a compact, battery-powered, and easily deployable three-axis actuator system suitable for mobile surgical environments.

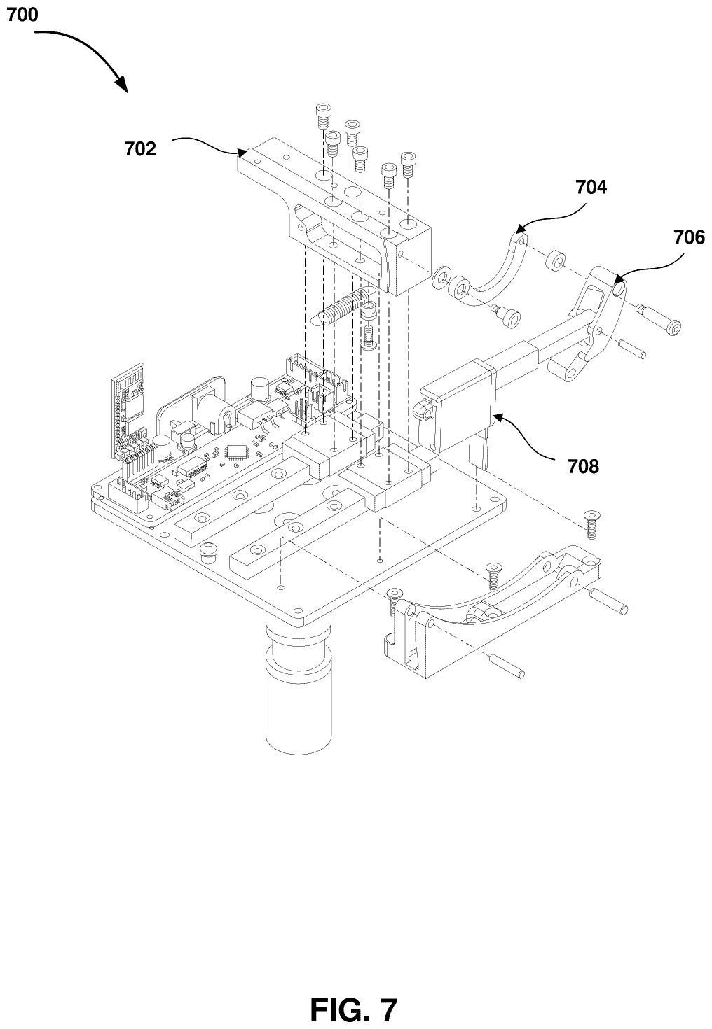

FIG. 7 illustrates an exploded assembly view 700 of a partially constructed actuation and linkage subsystem, which forms part of a three-axis actuator system for a portable microscope. This figure focuses on the integration of mechanical linkage elements, structural supports, and precision fasteners onto the previously established actuator baseplate. The uppermost component in view is the upper rail mount 702, a rectangular block that is attached to the substructure via a series of socket head screws. These screws pass through the upper surface of the rail mount and secure it to the underlying spring mount spacer, which is in turn affixed to the base using a precision shoulder screw. This rail mount structurally supports the bent linkage assembly 704 and serves as an anchor point for the connected actuation levers. The bent linkage 704 is secured using a nylon plastic washer and is coupled to the short actuator lever 706 via a shoulder screw. This arrangement allows rotational or pivotal movement between the two components, ensuring flexible transmission of mechanical input to the connected system. The short actuator lever 706 is further linked to a precision dowel pin and a camshaft body 708, which interfaces with a motion-translating mechanism such as a linear actuator or gear shaft.

A critical functional component in this arrangement is the P8_25M M_BODY and the associated P8_25M M_SHAFT, which serves as a coupling mechanism to convert rotary to linear motion or vice versa. The shaft is supported by a M 3-6 mm machine screw that threads through the base and secures the shaft to the internal assembly. This figure also depicts precision MGN linear rails and carriages (from the prior figure), which guide motion along a defined plane. Beneath the primary actuation cluster, the curved component represents the straight linkage, which is anchored to the base using cylindrical spacers and mounting pins. This part functions as a motion stabilizer or balance arm, translating forces from the vertical assembly into the horizontal plane and aiding in structural integrity.

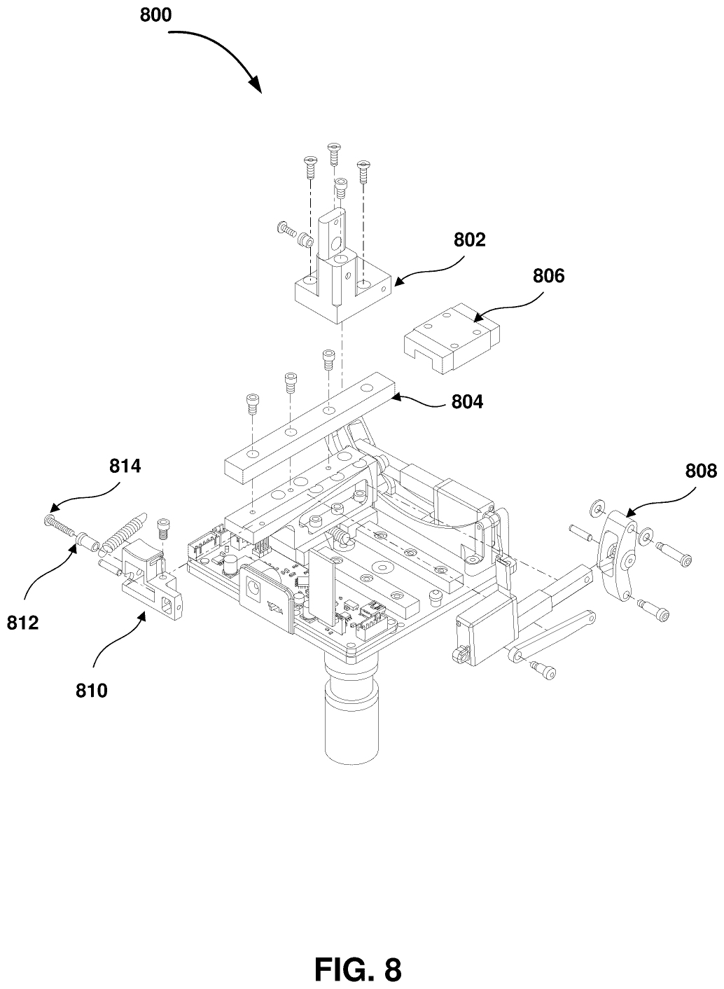

FIG. 8 provides an exploded perspective view 800 of the mechanical and control elements of the actuator subassembly, showing how upper rail structures, linkages, and mounting hardware interface with the electronic control system and linear motion platform. This figure particularly emphasizes the alignment and integration of the upper actuator mount and carriage mechanisms with the overall control and structural architecture. At the center of the assembly, the upper plate 802 is supported on spring mount spacers and is fastened to the underlying structure using multiple machine screws and button head drive screws. This upper plate 802 serves as the foundation for mounting the 12 mm linear rail 804, which is secured via a series of evenly spaced socket head screws. Riding along this linear rail 804 is the 12 mm carriage 806, which allows smooth translation along the guide axis and supports the dynamic motion of the connected actuator linkages. Attached to the right-hand side of the structure is the short actuator lever 808, which interfaces with other mechanical linkages and is mounted using a dowel pin and flat head screw. Adjacent to this is the bent linkage, which is again connected using a nylon washer to minimize friction and provide flexibility during angular motion. These two components cooperate to transmit mechanical force between the carriage and the vertically-oriented Z-axis actuator, allowing for coordinated multi-axis adjustments. To the left side of the assembly, the upper actuator mount 810 and the spring-loaded straight linkage 812 are shown in alignment. The actuator mount 810 is anchored using precision fasteners and includes a rectangular bracket that seats the handle grip or camshaft lever 814. This handle is threaded into position using a stainless steel shoulder screw and retained by an inner mounting block, which also supports a section of the rotating or pivoting mechanism. These components work together to stabilize the motion of the system during manual adjustment or in response to automated control inputs. The figure also shows the interconnection between the electronic controller platform and the linear rail structure. The controller platform contains pre-mounted circuit modules that manage RF signal reception and actuator power distribution. Mounting holes on the platform and rail assembly are precisely aligned to accommodate the rail using hardware such as button head and flat head screws, ensuring a stable and reliable interface.

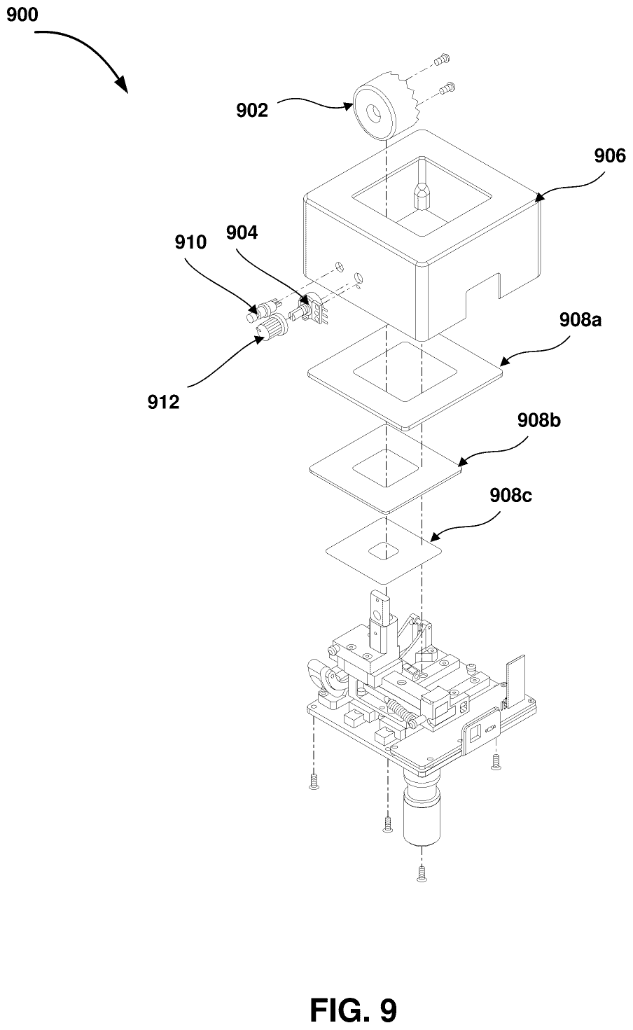

FIG. 9 illustrates an exploded assembly view 900 of a compact user-interface module integrated atop the actuator platform, emphasizing the enclosure, control knob interface, and multi-layer structural shielding. This portion of the assembly plays a dual role of both mechanical protection and user operability, and is essential in the practical use of the three-axis actuator for a portable microscope. At the top of the assembly is a turn knob 902, designed with tactile grip geometry and internally keyed for mounting onto a potentiometer shaft. The knob 902 is connected to a rotary potentiometer 904 via a retention fastener and is further secured by a hex screw or set screw, ensuring rotational alignment and axial stability. The potentiometer 904 itself is mounted to the side of a square cover enclosure 906, which houses the upper interface layer and provides a rigid shell to protect underlying electronics. The potentiometer's 904 purpose is to fine-tune sensitivity or gain for Z-axis or focus adjustments, allowing the user to make micro-modifications with analog precision. Below the enclosure cover 906 are three intermediate rectangular interface layers 908a, 908b, 908c. These layers are made from plastic, foam, or composite material and serve as electrical insulation, dust shielding, and mechanical dampers. Each sheet has a square central aperture and is dimensionally aligned to the enclosure housing. Their stacked arrangement helps absorb shock or vibration, prevents ingress of particulates, and electrically isolates the control interface from the actuation electronics. These layers also support the precise alignment of the potentiometer 904 within the enclosure 906 and reduce lateral play during knob rotation. To the left side of the enclosure, a momentary switch 910 is mounted adjacent to the potentiometer 904 and is further equipped with a small actuator button or toggling feature 912. This switch 910 provides a discrete digital input, such as a reset, mode toggle, or manual override, for controlling the actuator behavior. It is secured through the enclosure wall and wired into the internal PCB seen in prior figures. Beneath the multi-layer stack is the main actuator and PCB module, which serves as the base for this assembly. The cover, knob, and shielding layers are aligned above this platform, and fastened to it using a set of threaded screws extending through the enclosure corners. This ensures a firm mechanical connection between the control layer and the actuator subsystem.

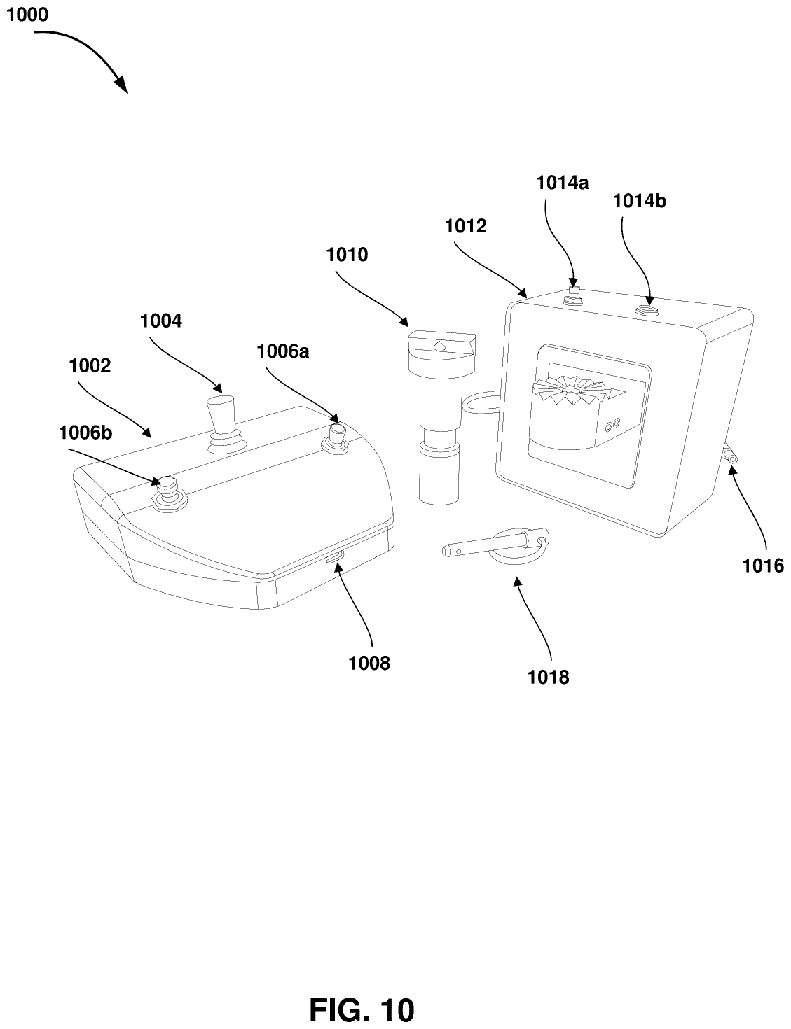

FIG. 10 illustrates a real-world, fully assembled setup of a three-axis actuator system 1000 designed for portable microscope manipulation. As shown, the wireless foot control module 1002, which comprises a central joystick 1004 for XY-axis navigation and two flanking SPST momentary push-button switches 1006a and 1006b that control up and down motion along the Z-axis. The enclosure is ergonomically shaped with a compact and low-profile design to support easy floor placement and intuitive foot operation. A USB port such as a USB-C port 1008 is visible on the front face, which is used for charging or firmware updates, emphasizing the device's self-contained, battery-powered operation. Further, the actuator shaft unit 1010, a cylindrical component with a stepped design and a precision-machined upper interface that mechanically couple with the microscope or an optical head. The compact form factor and metallic finish suggest durability and alignment accuracy during installation and use. Further, the main controller and housing unit 1012 are shown, enclosed in a robust rectangular casing. On the top surface, two tactile push-button switches 1014a and 1014b are present: one 1014a of which is marked for high-visibility, indicating a power or emergency stop function. The front face features a distinctive aperture design, a protective shroud for a rotating or optical component, with a series of embedded indicator LEDs aligned horizontally to provide real-time status feedback. The enclosure also includes a side power input jack 1016, confirming mains or external power compatibility. Further shown is a metal pin and ring 1018, which may be used to secure the actuator to a base or mounting interface, contributing to the portability and modular assembly of the overall system.

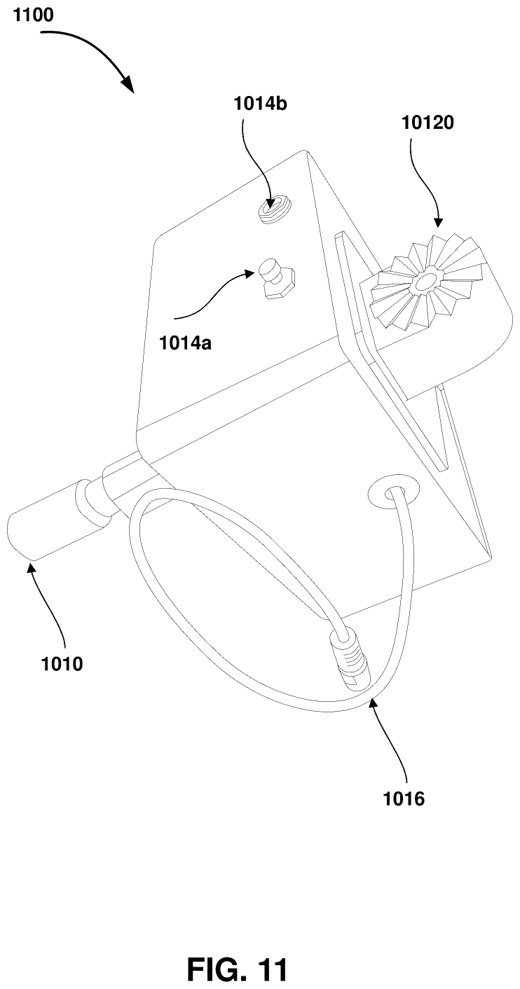

FIG. 11 depicts a close-up view 1100 of the assembled control housing of the three-axis actuator system, showcasing its angular enclosure, integrated actuation mechanism, and power interface. Prominently featured is the rotary component 1020 with a radial finned design extending from the front face of the enclosure. This component, part of the Z-axis focus actuator or a lens mount, exhibits a starburst-like geometry that suggests it either houses an optical element or is designed to engage with a precision mechanical interface for controlled movement. The radial symmetry provides enhanced grip and torque translation, which is critical for accurate micro-adjustments in a surgical or diagnostic setting. The top surface of the enclosure includes two tactile push-button switches 1014a and 1014b, with one accented by a colored cap such as a red cap, indicating a high-priority function such as emergency stop, reset, or power activation. The adjacent metallic button may serve as a standard control input. The angular shape of the enclosure not only accommodates internal circuitry and mechanical components but also aids in orienting the assembly within a workspace, maintaining cable clearance and visibility of interface elements. Emanating from the side of the enclosure is a power cable 1016, securely routed through a reinforced rubber grommet to ensure strain relief and insulation. This cable connects the actuator housing to an external power source, emphasizing the system's modularity and ease of deployment in varied operating environments. The rear cylindrical structure 1010 is consistent with the actuator mount or shaft interface, which couples the unit to a microscope stand or adjustable platform.

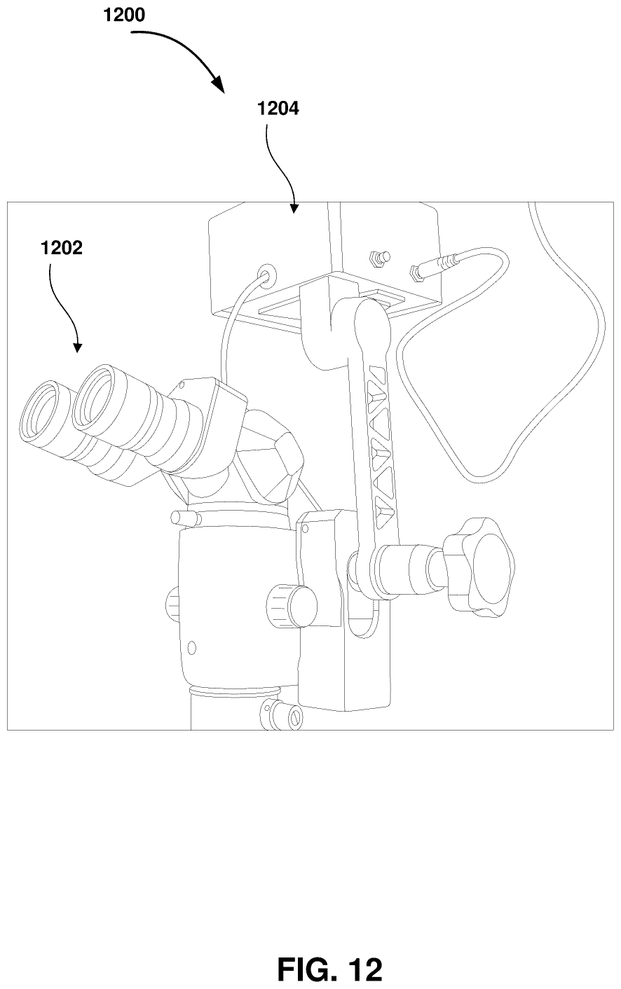

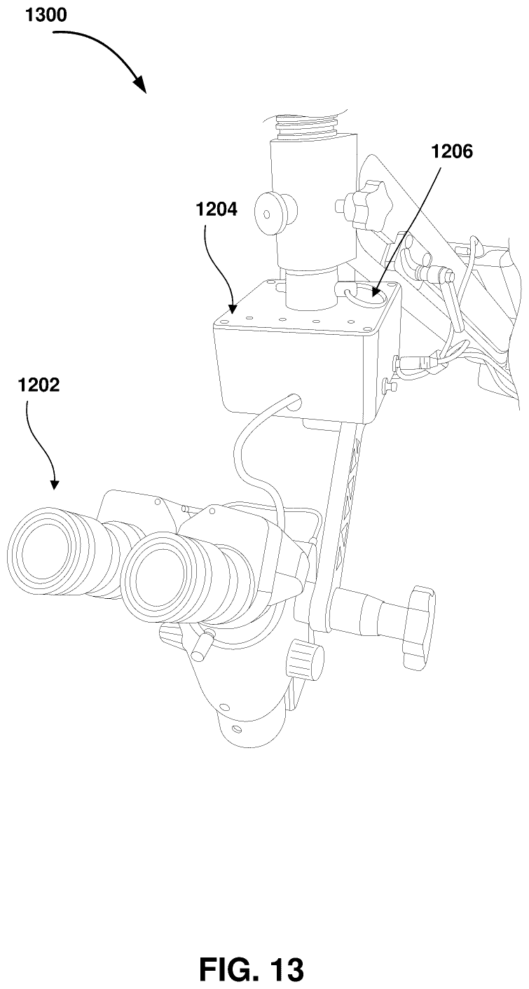

FIGS. 12 and 13 illustrate exemplary scenarios 1200 and 1300 of complete integration of the three-axis actuator system with an optical microscope, mounted on an articulating arm for clinical or laboratory use. At the center of the configuration is the actuator enclosure 1204, securely mounted above the microscope's objective tube. This enclosure houses the motion control electronics and mechanical actuation components that drive precise positioning of the microscope 1202 along X, Y, and Z axes. It is physically coupled to the microscope via a custom-fit interface or adapter, ensuring stability and accurate alignment during focus and repositioning tasks.

The microscope 1202 is a dual-eyepiece optical unit, equipped with two objective lenses that allow for stereo vision, making it suitable for surgical, dental, or micro-assembly applications. A pair of connection cables extend from the actuator enclosure, providing power and signal communication, one cable to be for power input, while the other may be for RF or control signal feedback. The enclosure also includes a ring-mounted clevis pin on top 1206, serving as a locking or release mechanism to enable quick installation or removal of the actuator system. The actuator is supported by a mechanical armature, in this case, a robust swing arm with multi-axis adjustability, which allows the microscope 1202 and actuator system 1204 to be positioned above a work surface. The black metallic finish of the actuator and arm complements the microscope body. The entire setup highlights the practical deployment of the actuator system in real-world use, demonstrating its compatibility with standard microscopes and its intended application in environments that require hands-free, high-precision focus and positioning control.

In conjunction with FIGS. 1-13, the operational functionality of a three-axis actuator system is designed for precise positional control of a portable microscope, wherein movement along X, Y, and Z axes is independently controlled through modular actuator assemblies. The system comprises two primary actuators: a planar XY actuator and a vertical Z actuator, each configured to translate control inputs into fine mechanical displacement. The XY actuator is operatively coupled to a structural platform supporting the microscope and is capable of lateral bidirectional movement along orthogonal axes, thereby enabling repositioning of the microscope field of view without manual intervention. This planar motion is driven by internal micro linear actuators that respond to wireless control signals, permitting hands-free adjustment with high spatial accuracy. The Z actuator is vertically mounted and mechanically linked to the microscope's objective lens assembly. Upon receiving an actuation signal, the Z actuator induces controlled linear displacement in the vertical direction, effectuating focal adjustments. The device facilitates and demonstrates seamless transitions between axial movements, evidencing precise and coordinated control across the three spatial axes. Movements are smooth, repeatable, and executed without perceptible delay or jitter, indicating the use of low-latency signal transmission and finely tuned actuation mechanisms. The actuation system is wirelessly controlled via a user interface, which may include a joystick and momentary switches, as previously disclosed. The modularity of the design is further apparent, as both X Y and Z assemblies operate independently while maintaining spatial stability of the microscope. The coordinated motion across axes allows for dynamic repositioning and focal calibration, which is particularly advantageous in surgical or diagnostic applications requiring real-time precision without manual disturbance of the optical system.

The present invention relates to a modular, wireless motion control system for an optical device, such as a portable microscope. M ore particularly, the system comprises an XY actuator module, a Z actuator module, and a wireless control interface that collectively enable precise, hands-free three-dimensional positioning and focal adjustment of the optical device in surgical, diagnostic, and field-based applications. The invention includes an XY actuator module that enables displacement of the optical device in a planar two-dimensional space, including a first direction (X-axis) and a second, orthogonal direction (Y-axis). The XY actuator module includes a pair of linear rails affixed to a base platform, with multiple pillow blocks and carriages facilitating linear motion in orthogonal directions. Integrated micro linear actuators are configured to drive these carriages in response to discrete actuation signals. A mounting arm or surface is operatively connected to the carriage structure and is adapted to support the optical device, such as a microscope or camera, such that the device can be translated in the horizontal plane without manual adjustment.

The system further includes a Z actuator module, configured to move at least a portion of the optical device in a vertical direction (Z-axis), which is orthogonal to the planar XY movement. In one embodiment, the Z actuator is directly connected to the objective lens assembly of the microscope. The Z actuator comprises a micro linear actuator housed within a rigid enclosure, a camshaft, and a coupling mechanism such as a pin or shaft, which converts linear motion into rotary or vertical translation. The Z actuator provides precise focal adjustment of the microscope without moving the entire microscope body, significantly reducing mass, inertia, and energy requirements. A wireless control interface is used to operate both the XY and Z actuator modules. In one embodiment, the interface is realized as a foot-operated control module, comprising a joystick and two SPST (Single Pole Single Throw) momentary push-button switches. The joystick is configured to transmit directional control signals for X and Y movement via integrated radio frequency (RF) transmitters, while each push-button switch corresponds to Z-axis upward or downward actuation. These control signals are received wirelessly by onboard receivers embedded within the actuator modules. This configuration enables real-time, user-directed motion across all three axes without requiring hand contact, thereby supporting sterile-field compatibility in surgical environments.

The actuator modules are powered either by an external DC power source or by internal portable batteries housed within the system or the control module itself. The system supports low-latency, discrete command-based actuation, which ensures precise motion with minimal delay and eliminates the need for analog voltage regulation or high-bandwidth data links. The system architecture is modular, wherein the XY actuator module and the Z actuator module may be used independently or in combination. Both modules are designed with quick-mount interfaces, allowing the invention to be adapted across a variety of microscope models or custom imaging platforms. The housing of each module is constructed from lightweight materials such as aluminum alloy, minimizing system weight while maintaining structural rigidity. Mounting brackets, spacers, and vibration-isolation washers are used to enhance mechanical stability and repeatability.

In operation, the user may interact with the wireless control interface to initiate motion in the desired direction. For XY movement, the user engages the joystick in the appropriate direction (e.g., North, East, South, West, or diagonal orientations), resulting in movement of the microscope platform along the horizontal plane. For Z-axis control, the user activates either the upward or downward push-button switch to drive the objective lens closer to or farther from the specimen plane. The use of discrete RF signals simplifies the control protocol and avoids the complexity of analog modulation or digital pairing. The system is particularly well-suited for use in portable, battery-powered medical imaging scenarios where space, weight, and hygiene are critical concerns. The compact design enables transport in a small protective case and allows for rapid deployment in field hospitals, rural clinics, or mobile surgical units.

While various embodiments of the disclosure have been illustrated and described, it will be clear that the disclosure is not limited to these embodiments only. Numerous modifications, changes, variations, substitutions, and equivalents will be apparent to those skilled in the art, without departing from the spirit and scope of the disclosure, as described in the claims.

What is claimed is:

1. A motion control system for positioning an optical device, comprising:
an XY actuator configured to displace the optical device in a first planar direction and a second planar direction orthogonal to the first planar direction;
a Z actuator configured to displace at least a portion of the optical device in a direction perpendicular to the first and second planar directions; and
a wireless control interface configured to transmit control signals to the XY actuator and the Z actuator to independently actuate each actuator.

2. The system of claim 1, wherein the XY actuator comprises a pair of linear actuators and a platform for supporting the optical device.

3. The system of claim 1, wherein the Z actuator is configured to adjust the position of a lens or objective component of the optical device to perform a focal adjustment.

4. The system of claim 1, wherein the wireless control interface comprises a joystick configured to control displacement along the planar directions and one or more momentary switches configured to control displacement along the perpendicular direction.

5. The system of claim 1, wherein at least one of the XY actuator or the Z actuator comprises a micro linear actuator responsive to wireless signals.

6. The system of claim 1, wherein the XY actuator and the Z actuator are mechanically decoupled and operable independently.

7. The system of claim 1, wherein the XY actuator and the Z actuator are modular and removably attachable to the optical device or a support structure coupled to the optical device.

8. The system of claim 1, wherein the actuators are powered by a portable battery housed within the system or the wireless control interface.

9. The system of claim 1, wherein the system enables repositioning and focal adjustment of the optical device without physical contact between a user and the optical device.

10. The system of claim 1, wherein each of the XY actuator and the Z actuator comprises a structural housing, an actuator motor, and a mounting interface configured to couple with the optical device or its support structure.
